(19) United States
(12) Patent Application Publication
(10) Pub. No.: US 2018/0164278 A1
(43) Pub. Date: Jun. 14, 2018

(54) TESTING ARRANGEMENT FOR EXAMINING A CELL CULTURE UNDER THE EFFECT OF A DYNAMIC FORCE

(71) Applicant: TECHNISCHE UNIVERSITÄT DARMSTADT, 64289 Darmstadt (DE)

(72) Inventors: Tobias MECKEL, Darmstadt (DE); Eva SAPPER, Mörlenbach (DE); Ljubomira Ana SCHMITT, Pfungstadt (DE)

(73) Assignee: TECHNISCHE UNIVERSITÄT DARMSTADT, 64289 Darmstadt (DE)

(21) Appl. No.: 15/581,000

(22) PCT Filed: Jun. 8, 2016
(86) PCT No.: PCT/EP2016/063082

§ 371 Date: Dec. 8, 2017

(30) Foreign Application Priority Data
Jun. 9, 2015 (DE) 10 2015 109 087.2

(51) Int. Cl. G01N 33/483; C12M 1/12; C12M 3/00; C12M 1/00; G01N 3/08

(52) U.S. Cl. CPC G01N 33/4833; C12M 25/14; G01N 3/08; C12M 23/22; C12M 23/48

(57) ABSTRACT

A test arrangement for examining a cell culture under the effect of a dynamic force has a three-dimensionally designed support structure which is designed such that the cell culture is embedded into the support structure. By applying a force, the support structure is deformed, wherein a force application device has a first actuator which acts on the support structure at a distance from the cell culture embedded in the support structure. The force application device has a second actuator and a third actuator, each of which acts on the support structure at a distance from the cell culture embedded in the support structure. The third actuator exerts a force onto the support structure, the force being oriented differently than the force exerted by the first actuator and the force exerted the second actuator.

### BACKGROUND AND SUMMARY

The invention relates to a testing arrangement for examining a cell structure under the effect of a dynamic force with a support structure for admission of a cell culture and with a force admission device.

Many fundamental concepts for intra-cellular information transmission and transmission between cells and their environment are done on cell cultures which are bred and cultivated outside a living organism “in vitro,” for example in a Petri dish. The cell cultures produced and examined “in vitro” regularly exhibit a planar, two-dimensional arrangement of a large number of cells, which form a cell culture. Individual cells as well as the entire cell culture can be examined and researched with the two-dimensional arrangement of the cell culture in a simple way with optical microscopes or with other suitable measuring devices.

It has been shown that the behavior and the examination results of an essentially two dimensional cell culture cannot be readily transferred, to living organisms, in which three-dimensional cells linked with each other form a natural biological system or tissue structures. It is known that through an arrangement of the body's own cells on suitable support structures, and their deliberate proliferation and linking “in vitro,” stable tissue structures can be generated, which for example form a substitute tissue which as an artificial connective tissue or epithelial tissue is suitable for implantation.

Further investigations have shown that individual aspects such as cell growth, cell proliferation, cell migration and cell differentiation are all complex processes which depend on intracellular factors and extracellular factors. For example, along with molecular and biochemical signals and environmental conditions, also mechanical forces and electrical signals can have an effect on the development of cells.

Further it has been shown that the cell behavior of individual cells in a two-dimensional arrangement differs markedly from the cell behavior of comparable ceils in a three-dimensional cell structure, which is generated and cultivated in a suitable support structure. For example, the mechanical. rigidity of the support structure among other factors has an effect on mechanical sensor technology, cell adhesion and cell migrations and also influences the diffusion of dissolved substances and the binding of proteins, so that in dependence on the support structure, certain cells are influenced in their morphogenesis and proliferation.

To be better able to investigate the influence of the various normally employed support structures and environmental conditions on cell cultures, the cell culture during cultivation, can be subjected to the effect of a dynamic force. For example in US 2014 038 258 A, a bioreactor is described in which, in a cell culture placed in a nutrient solution, electrical stimulation as well as mechanical force can be exerted. The mechanical force is generated by a movable piston, which acts on a thin, deflectable membrane. On the surface of the thin membrane, cells grow which can be stretched by the deflection of the membrane.

In US 2013 059 324 A, a similar action principle is described in a microtiter plate for investigation of a large number of cell cultures in which, in the individual cavities of the microtiter plate, cells are cultivated on the surface of a deflectable, stretchable membrane. With a stamp plate adapted to the microtiter plate, in all cavities the membrane and thus the particular two-dimensional cell culture can be subjected to this same stretching.

In DE 101 51 822 A1, a device is described for electrical and mechanical stimulation of cells that are applied to the surface of a support, and. the support is held by movable clamps in a culture chamber and subjected to a mechanical loading by deflection generated by a motor of the clamps. Due to the configuration of the device, and especially of the movable clamps, an optical investigation and observation of the cells can be conducted only from a great distance therefore with an enlargement of the optical imagery limited to 10 to 20 times.

The devices known from the practice often make possible only an application of force on a planar or two-dimensional cell arrangement, which for example is applied to a deflectable, stretchable membrane or on a mechanically loadable support. In most cases a force is exerted only in one direction on the planar cell arrangement, so that for example, due to the elasticity of the deflected membrane, it resets into an initial position. To be able to generate the desired force application, as a rule structurally expensive devices are required, which impair or make impossible simultaneous studies of planar cell arrangements. Therefore, for examination, a cell structure must be removed from such a device and transferred to a suitable examining apparatus.

It is desirable to so configure a testing arrangement to examine a cell culture under dynamic application of force, so that not merely two-dimensional cell cultures arranged on the surface of a support structure can be subjected to mechanical forces, that the application of force can be controlled as precisely as possible, and that also an investigation of the cell culture can be carried out during the application of force.

In accordance with an aspect of the invention a support structure is configured to be three-dimensional and so configured that the cell culture is embedded into the support structure, wherein through an application of force the support structure can be deformed, and that the force application device has a first actuator, which acts on the cell culture embedded hi the support structure at a distance from the support structure. The support structure can for example be produced from a suitable elastomer and by means of polymerization or with the aid of a three-dimensional compression process be configured as a three-dimensional scaffold structure. The cell culture is embedded in the support structure in such a way that through a forced deformation of the support structure a force application is exerted on the cell culture embedded in the support structure.

In advantageous fashion a deformable elastic or fiber-containing structure can be made of a suitable polymer such as silicon or latex, or consist of collagen or gelatin, or have these materials in combination with additional support materials. It is also possible that a natural tissue such as myocardium tissue or an artificial biological tissue material can be used as a support structure. Still other suitable support structures are described in what follows. The support structure can be treated by suitable methods such as copolymerization, plasma treatment, an etching process or irradiation, as well as with the aid of chemical or biological substances, to promote adherence of the cells of the cell culture on the support structure.

The force application device acts on the support structure using a first actuator at a distance from the cell culture embedded in the support structure. The force is applied consequently in a way and means that is modeled on the relationships in native cell tissues and cell environments and therefore makes possible realistic and significant examination results.

The actuator that is situated at a distance from the cell culture makes possible mechanical loading of the cell culture and simultaneously allows an examination of the mechanically loaded cell culture, for example with high-resolution optical microscopes or fluorescence microscopes. Through a suitable configuration of the support structure, what can be attained is that the support structure, with usage of the actuator, is deformed over a large spatial area, so that a correspondingly large actuator distance from the cell culture can be preset. Due to the actuator being at a great distance from the cell culture, an analytic device such as an optical microscope can be brought very close to the support structure, and going along with this, very close to the cell culture to be examined, so that for example it is possible to use a microscope lens with 100× magnification with a numerical aperture of more than 1.4.

According to an advantageous configuration of the invention concept, provision is made that the force application device has a second actuator which acts on the support structure at a distance from the cell culture embedded in the support structure, and that the first actuator and the second actuator exert directed forces that differ from each other on the support structure. For example, the first actuator and the second actuator can be aligned perpendicular to each other, or generate a force application directed perpendicular to each other. In this case, with the first actuator and the second actuator, through a suitable superimposition of the particular force application, any force generation directions can be produced in a force application plane preset fey the first actuator and the second actuator, and imposed on the cell culture.

The first actuator and the second actuator can be operated in different ways and means, and independently of each other. For example, by the first actuator a cyclic compression can be exerted with a presettable frequency on the support structure and thus on the cell culture, while with the second actuator, a constant pressure loading, or one that changes at markedly greater time intervals can be exerted on the support structure or on the cell culture. The directions of force exertion of the first actuator and the second actuator can also have angles relative to each other of between 0° and 180°.

Especially preferred is a provision that the force application device has a third actuator, which likewise acts on the support structure at a distance from the cell culture embedded in the support structure, and that the third actuator exerts a force application on the support structure deviating from that of the first actuator and from the second actuator. Through a suitable arrangement and direction of the three actuators, virtually any directional forces acting on the support structure can be generated. Additionally, the resulting total force that acts on the cell structure can be precisely preset and reliably exerted with the aid of the three actuators in all spatial directions. Through the use of three actuators which impinge in differing directions on the support structure, complex loadings on the cell culture that closely approximate real life can be simulated and be evaluated with examinations that coincide in time or are carried out subsequently.

To be able to exert a force application that is able to be preset as precisely as possible, with environmental conditions that can be controlled and reproduced as well as possible, provision is made that the trial device has a holding rack to admit the support structure, and that at least one of the actuators is attached on the holding rack in such a way that the actuator can exert a force that deforms the support structure on the support structure admitted in the holding rack.

According to an advantageous embodiment of the invention concept, provision is made that the actuator fixed on the holding rack is in effective connection with a first side of the support structure, and that the support structure on a second side that is opposite to the first side. is secured to an attachment-device on the holding rack, so that via the actuator, tensile forces or compressive forces can be transmitted to the support structure. For example, the support structure can be secured by clamping in the attachment device on the holding rack. It is also possible to secure the support structure with a suitable gluing agent or adhesive agent on an adhering surface of the attachment device. Also conceivable, and advantageous for certain instances of application and support structures is form-locking securing of the support structure on the attachment device.

With an actuator that is adjustable in linear fashion, precisely presettable tensile forces or compression forces can be transmitted to the support structure in a simple way. Along a preset direction, actuators that in essence have one-dimensional adjustment capacity, which have a sufficiently long adjustment path and a sufficiently powerful actuating drive, are obtainable at reasonable cost in trade in numerous versions.

It is also possible that the attachment device on the second side of the support structure has an additional counter-actuator, which is secured to the holding rack and likewise can exert a force on the support structure. Like the first actuator, which is situated on the first side of the support structure and acts on the support structure, the additional counter-actuator can exert an essentially linear force on the support structure, that either is in a direction opposite to, or is rectified to, the force application of the first actuator. With a simultaneous force application in the opposite direction, an enhanced compression or stretching of the support structure is brought about.

With a rectified force application, the support structure is displaced and accelerated by the two actuators, the first actuator and the assigned counter-actuator, so that the cell culture embedded in the support structure is subjected to all the acceleration forces that are generated by displacement of the support structure. As with the first actuator, also the second actuator and the third actuator can each be combined with attachment devices situated on opposite sides. The attachment devices can also each have ant additional counter-actuator. To make possible as comprehensive a control and as precise a presetting as possible of mechanical loading of the cell culture, in all three spatial directions on each two opposite sides of the support structure, an actuator and a counter-actuator can be so situated that with these actuators at a distance to the cell culture, a force application that deforms the support structure can be generated. Also, use of less than six actuators or of more than six actuators can be appropriate for examinations of cell cultures with three-dimensional force applications, simulated as realistically as possible.

To simultaneously be able to apply a mechanical loading and to conduct an optical examination of the cell culture, provision is made that the holding rack is configured as a frame rack, and that at least on two opposite rack sides, optically transparent openings are found, through which the support structure can be illuminated and observed. The holding rack for example can be configured as a grid cage and make possible from all sides a virtually unobstructed view of the support structure and the cell culture embedded in it. It is likewise possible that the holding rack be composed of multiple rods connected with each other, which form a truss. In regard to a possibly desired screening of the support structure, it can be advantageous if the holding rack has a housing that is closed cm virtually all sides, that surrounds a measurement and examination chamber. In which the support structure can be subjected to mechanical loadings and examinations can be carried out on the support structure.

The holding rack can also have a liquid-sealed sample chamber to admit the support structure with the cell culture. The actuators can be situated in an inner space of the sample chamber and be attached on chamber walls of the sample chamber or on holding structures provided for this. It is likewise conceivable that the actuators are situated outside the sample chamber and are in effective connection with the support structure via sealed openings that are in the sample chamber. To facilitate high-resolution optical imagining and examination of the cell culture, a microscope lens can be situated on or in the sample chamber, which is connected, or can be connected, with an optical imaging device or analysis device.

In an advantageous way, provision is made that, the first actuator and if necessary additional actuators and/or counter-actuators are supported so as to shift on the frame rack. The actuators mid if necessary counter-actuators that are supported so as to shift can be adapted with differing dimensions in regard to their particular positioning on the support structures. Additionally, a support structure held by the actuators and if necessary counter-actuators can in force-locked or form-locked fashion be shifted for its part by a shifting of the actuators and if necessary counter-actuators, to be moved for example automatically from a cell cultivation position in a nutrient solution into an analysis position, or into an analysis device.

According to an especially advantageous embodiment of the invention concept, provision is made that the support structure have at least one recess into which the first actuator can engage, to cause a deformation of the support structure by force application. Through creation of a recess in the support structure, the actuator can for example have a rod-shaped or lance-shaped actuator section, that thorough a linear displacement of the rod-shaped or lance-shaped actuator section can be inserted into the recess and if necessary also removed from the recess. Via the recess, in a simple fashion a form-locked connection can be achieved of the support structure with the actuator, and be maintained throughout the duration of the force application.

In appropriate fashion, provision is made that the support structure have at least two recesses, which are arranged at an angle, separated from each other, so that a first actuator and a second actuator can each engage on opposite sides of the cell culture in an assigned recess, to engage the support structure and to stand out from a background. Through an arrangement of recesses that is not parallel, but rather at an angle to each other, the first actuator and the second actuator with rod-shaped actuator sections or with engagement elements adapted to the configuration of the recesses can be made to engage so that the support structure can also be engaged with only two actuators and be able to stand out from a background, to implement a plurality of different force engagements. In addition, the support structure can also engage with the force application device and be shifted, for example to automatically move from a storage or supply device into a cultivation device or into an analysis device, or into the testing arrangement.

To be able to produce the recess simply and cheaply, provision is made that the at least one recess is configured like a pocket in the support structure.

With the invention-specific testing arrangement, using the force application device, during the cultivation of cells and during the examination or analysis of cells, a force acting in whatever directions on the support structure and thereby on the cells embedded therein can be generated. In regard to an efficient analysis of a large number of cells and cell cultures, an advantageous embodiment of the invention concept is consequently provided, that the holding rack has multiple force application devices each with at least one first actuator and multiple support structures, wherein with the multiple first-actuators a force application that deforms at least one assigned support structure is exerted on the support structure in question. Through the arrangement of a first actuator and a second actuator, as well as a third actuator if necessary, and additional actuators that are situated opposite each of these actuators, which are assigned to a support structure, forces can be simultaneously applied to each support structure, and if necessary independently of each other, from all directions, and thereby complex deformations of the support structures in question be generated by force. In this way a large number of support structures can be cultivated simultaneously and analyzed, and during this period of treatment, be subjected to whatever forces.

To make possible the use of microtiter plates, provision is made that the holding rack have a connection device for connection with a microtiter plate, so that the holding rack can be connected with the microtiter plate so that each support structure is arranged in an assigned cavity of the microtiter plate and can be deformed by the first actuator assigned to the support structure in question. The connection device can also have a receiving device, for example a pigeonhole or a drawer, for admission of a microtiter plate.

According to one embodiment of the invention concept, a provision is made that at least one of the actuators is a piezoactuator. From the practice, piezoactuators with a piezoceramic are known, by which deflections of up to 2 mm with short response times of about 20 milliseconds and a low operating voltage of less than 50 volts can be implemented. Such piezoactuators can be provided with controls and excursions over long intervals, so that over a long period they can exert a more precisely producible force application onto the support structure.

It is also conceivable that at least one of the actuators has a shape memory material. With the aid of shape memory alloys or shape memory polymers, mechanical deflections of an actuator valve can be stimulated for example electrically or thermally. It is also conceivable that at least one of the actuators is a polymer actuator operable in electrothermal fashion.

To be able, using the actuators, to document forces and mechanical loadings on a low structure, provision is made that at least one sensor device is arranged in the actuators and/or in the support structure to detect, forces acting on the support structure. The sensor device can for example have a position sensor or a path sensor and detect a relative or absolute displacement or deformation of the support, structure, so that, via reference measurements and comparison values, the force acting on the support structure can be determined. It is also possible to arrange miniaturized pressure or force measurement sensors on or in the support structure.

From the practice, elastomers are also known, in which a compression or a stretching of the elastomer can be optically detected.

It is also possible to arrange and secure at least one actuator at a distance to the cell culture on the support structure so that the activation of the actuator causes a deformation of the support structure. Electroactive polymers are known in which, through the application of a altering current or altering charge, the shape of the electroactive polymer can be changed. Counted as being among these are both ionic electroactive polymers such as conducting polymers, ionic metal-polymer composites, or ionic gels and also electronic electroactive polymers such as, for example, electrostrictive and ferroelectrical polymers as well as dielectric elastomer actuators. With such electrically active polymers, planar actuator strips, for example, can be generated, which can be arranged at a distance to the cell culture on the support structure and can be connected flat. By application of a suitable alternating current the actuator strip can alternately expand and contract, which immediately is transmitted to the support structure: connected therewith and forces a corresponding stretching or contraction of the support structure. The actuator strip is exclusively connected with the support structure, without requiring a holding rack to secure the actuator strip. The electrically active polymer can also have a shape deviating from a strip, which is adapted to the desired deformation of the support structure.

In an advantageous manner provision is made that multiple actuators surround the cell culture to form a frame on one surface of the support structure. By this means, with a suitable operation of the actuators, longitudinal and lateral contractions can be induced into the support structure in an area surrounding the cell culture, which can be transmitted through the support structure to the cell culture. Due to the framelike arrangement, a uniform and controllable deformation can be exerted on the area of the support structure surrounded by the framelike arrangement.

The actuators can, for example, be compressed by suitable compression procedures onto the support structure. It is also possible to connect or adhesive-bond the actuators onto the support structure in force-locked or form-locked fashion. With a support structure having a three-dimensional structured surface, a form-locked connection of the actuators with the support structure can also be implemented. The support structure can also have a strip-shaped recess for example, into which the assigned actuator is embedded.

Along with electrically active polymers, numerous other actuators or active mechanisms are known which are likewise suited to either be connected with the support structure in combination with a holding rack, or exclusively with the support structure, and when operated, effect a deformation of the support structure. Thus for example, electromechanical, electrochemical, magnetostrictive, hydraulic or pneumatic, bimetal or also electromagnetic actuators, such as voice-coil actuators, can be used.

In advantageous fashion, provision is made that the support structure has fibers. The fibers can transmit tensile forces in the fiber direction over large areas, and with a suitable connection effect of the fibers among each other, far over one fiber length. A support structure basing fibers can be deformed extensively in simple fashion through forces acting from the outside. Between individual fibers, the cell culture can be embedded in the support structure.

The material used for the support structure can be configured to be porous, and facilitate a simple admission of nutrient solutions and dissolved chemical or biological substances to the cell culture. The material can be sufficiently transparent for optical examinations, so that a microscopic examination of the cell culture embedded in the support structure is possible in nearly unobstructed fashion. A large number of different materials with varied properties is obtainable in regular commerce and is inexpensive.

According to an especially advantageous embodiment of the invention concept, provision is made that a biocompatible hydrogel is embedded into the support structure to admit the cell culture.

The hydrogel can form a matrix in the support structure, in which the cell culture is embedded, A suitable hydrogel can for example be a collagen, gelatin or a polyethylene glycol. The hydrogel can also have laminin, fibronectin or hyaluronic acid or essentially consist thereof. The hydrogel can transmit the force of the support structure deformed by the actuators to the cell culture. Through the hydrogel, advantageous environmental conditions can be preset for the cultivation of the cell culture.

### BRIEF DESCRIPTION OF THE DRAWINGS

In what follows, embodiments of the invention concept are explained in greater detail, shown in the drawings. Shown are:

FIG. 1: a schematic perspective illustration of the testing arrangement with a cuboid support structure, which is secured with actuators in a holding rack surrounding the support structure.

FIG. 2; a schematic sectional view of the testing arrangement in FIG. 1 along the horizontal plane II-II in FIG. 1.

FIG. 3: a schematic sectional view of an alternately configured testing arrangement, in which a holding rack is arranged on a microtiter plate, in which multiple support structures are arranged and can be deformed by multiple force admission devices.

FIG. 4; a top-down view of the testing arrangement shown in FIG. 3.

FIG. 5; a schematic sectional view of a support structure, which is held by a force admission device with a first actuator, a second actuator and a third actuator.

FIG. 6: a schematic lateral view of the support structure shown in FIG. 5, with the assigned force admission device.

FIG. 7: a schematic view of a planar cuboid support structure, on which four actuators are arranged in frame fashion.

FIG. 8: a top-down view of the testing arrangement shown in FIG. 7.

FIG. 9: a schematic sectional view along the line IX-IX in FIG. 8, and

FIG. 10: a schematic sectional view as per FIG. 9, wherein the actuator, differing from the embodiment shown in FIG. 9, is attached to the support structure.

### DETAILED DESCRIPTION

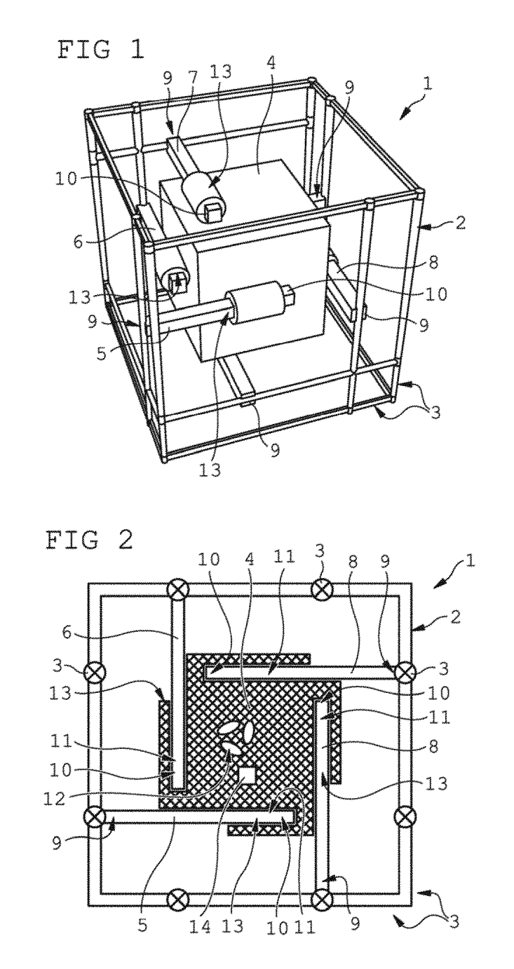

A testing arrangement 1 depicted schematically in FIGS. 1 and 2 has a holding rack 2 which is composed of multiple rods 3 that connect with each other at right angles and cross each other. In rack 2, a support structure 4 is arranged, which is supported in holding rack 2 by a first actuator 5, a second actuator 6 and a third actuator 7, as well as by three additional counter-actuators 8. The first actuator 5, the second actuator 6 and the third actuator 7 are arranged at right angles to each other, so that via these three actuators 5, 6 and 7, forces can be exerted from and in any three-dimensional direction on support structure 4. To each actuator 5, 6 and 7, a counter-actuator 8 is assigned, which is engaged on a side of support structure 4 that is opposite the actuator 5, 6 and 7 in question with support structure 4, and can either exert a rectified or opposite-directed force admission onto support structure 4.

Actuators 5, 6 and 7 and counter-actuators 8 are braced on a particular first end 9 of actuators 5, 6 and 7, as well as counter-actuators 8 on an assigned rod 3 of holding rack 2. Actuators 5, 6 and 7, as well as counter-actuators 8 are configured to be beam-shaped and each have an adjustment section 11, which starts at a distance from the first end 9 and extends to a second end 10 that is opposite first end 9, which, through an operation of the actuator 5, 6 or 7 or of counter-actuator 8, can be deflected or deformed in a preset way. By operating actuators 5 and 6, a force is exerted onto support structure 4, which is connected to transmit force in the area of adjustment section 11 with the individual actuators 5 and 6.

Each actuator 5, 6 and 7, forms an actuator pairing with its assigned counter-actuator 8, with which primarily in a spatial direction preset by the arrangement of the actuator pairing, a force can be exerted on support structure 4. The three actuator pairings are aligned perpendicular to each other and are so arranged that with the three actuator pairings, tensile and compression forces can be exerted in all three spatial directions on support structure 4. Holding rack 2 formed by rods 3 connected with each other exhibits a mechanical rigidity sufficient for this. At the same time, holding rack 2 covets support structure 4 arranged therein only in inconspicuous fashion, so that a cell culture 12 embedded in support structure 4 can be viewed, analyzed and examined almost unimpeded.

Through actuators 5, 6 and 7 as well as through counter-actuators 8, support structure 4 is supported in holding frame 2, wherein through an operation of actuators 5, 6 and 7, and of counter-actuators 8, a deliberate and controllable force can be exerted on support, structure 4, so that support structure 4 is deformed. The three actuators 5, 6 and 7 as well as counter-actuators 8 can also be supported so as to be movable on holding frame 2, so as to make possible a shifting of support structure 4 held by actuators 5, 6 and 7 and by counter-actuators 8.

To make possible the form-locking engagement, detachable if necessary, of actuators 5, 6 and 7 as well as counter-actuators 8 with support structure 4, support structure 4 has an assigned, pocket-shaped recess 13 for each actuator 5, 6 and 7, as well as counter-actuator 8. The two rod-shaped, or lance-shaped ends 10 engage into the pocket-shaped recesses 13. By shifting of support structure 4 or the actuators 5, 6 and 7 as well as counter-actuators 8, individual actuators 5, 6 and 7, as well as the counter-actuators S, or all of them, can be engaged into or disengaged from support structure 4.

Through the forced deformation by actuators 5 and 6 of support structure 4, a mechanical force is exerted on cell cultures 12 embedded in support structure 4. Support structure 4 consists of a material containing fibers, in which the cell cultures 12 are embedded. It is likewise conceivable with the aid of suitable shaping processes such as selective laser sintering, melt layering or three-dimensional compression techniques to produce a three-dimensional scaffold structure from a suitable plastic, from a ceramic or metal. With this, essential properties such as mechanical rigidity, pore size or porosity or the surface properties can deliberately be present and adapted to the trial requirements as well as to the cell cultures to be examined.

In support structure 4, in addition to the cell cultures 12, a sensor device 14 is also embedded, by which the force exerted by actuators 5 and 6 on support structure 4 can be detected.

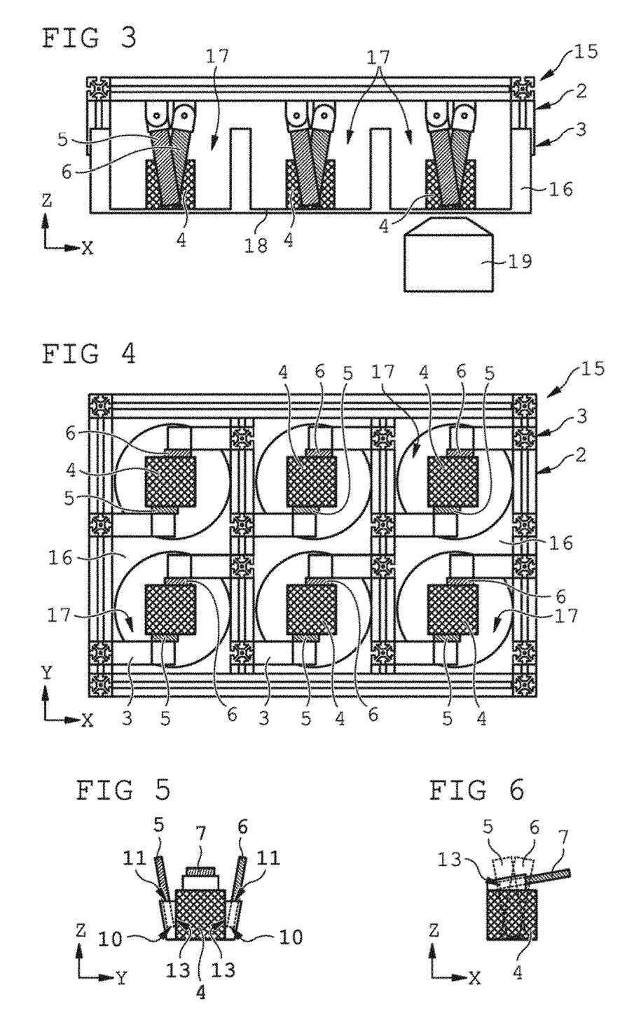

FIGS. 3 and 4 are examples of a testing arrangement 15 that is differently configured Holding rack 2 that is likewise assembled from rods is connected by a microtiter plate 16 with six cavities 17 separated from each other. For each cavity 17, in which a support structure 4 is found, the testing arrangement has an assigned force admission device with a first actuator 5 and a second actuator 6, which are aligned at an angle to each other on opposite sides of support structure 4 and each of which engages into a pocketlike recess 13 of support structure 4. Through the arrangement and alignment of first actuator 5 relative to second actuator 6, support structure 4 can be positioned or secured in space. With first actuator 5 and second actuator 6, the particular support structure 4 can consequently not only be deformed, but also engaged and lifted or displaced. Second actuator 6 can act like a counter-actuator 8 of the version depicted in FIGS. 1 and 2.

With testing arrangement 15, at the same time six different cell cultures 12 can be cultivated and analyzed in an assigned support structure 4, wherein at any time nearly any force can be excised on the individual support structures with the particular assigned force admission device, or with the assigned actuators 5 and 6.

To make possible an especially high-resolution and reliable examination of cell cultures 12, provision is made that microtiter plate 16, at least in the area of the cavities 17, has an optically transparent bottom area 18, so that a lens 19 of an optical examination device not depicted in greater detail, can be situated very close to support structure 4 and on cell culture 12 situated therein. In this way, for example, optical images can also be taken with 100× magnification, while a force is being exerted on cell culture 12.

A comparable testing arrangement can be provided and adapted for use with microtiter plates, which if necessary has considerably more cavities, for example 48, 96 or 384 or more or fewer cavities.

FIGS. 5 and 6 are two exemplary side views of a support structure 4, which is held by a first actuator 5, a second actuator 6 and a third actuator 7. The three actuators 5, 6 and 7 form the force admission device for this support structure 4. Through actuators 5 and 6 which are not situated parallel to each other, but at an angle relative to each other, support structure 4 can be engaged and positioned. Actuator 7, depicted above in FIGS. 5 and 6, can also generate or amplify a force on support structure 4, which is exerted in a direction designated as the Z axis in FIGS. 5 and 6. Such a force admission device can be implemented In each cavity 17 of testing arrangement 15. Especially actuators 7 can be supported so as to shift on holding rack 2, if necessary and for example during optical examinations by lens 19, to be withdrawn from pocket-shaped recess 13 of support structure 4.

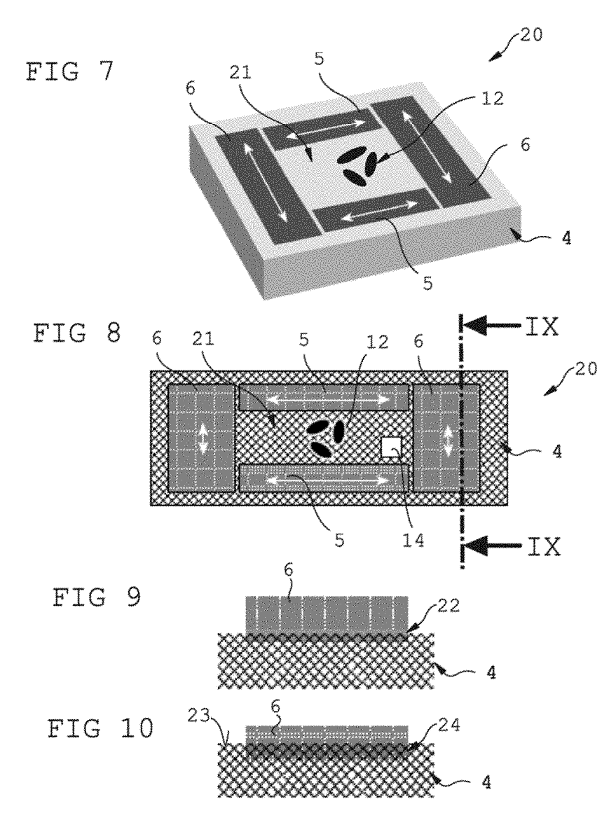

FIG. 7 is a schematic view of yet another alternative embodiment of testing arrangement 20 with a support structure 4 that is planar and cuboid shaped, on which two first actuators 5 and two second actuators 6 are arranged. These two first actuators 5 and the two second actuators 6 are planar, strip-shaped, electrically active polymeric layer actuators. Through a varying tension, the electrically active polymeric layer of actuators 5, 6 can be forced to expand or contract, which primarily causes a change in length in the longitudinal direction of actuators 5, 6. Through a temporally and spatially coordinated operation of the two first actuators 5 and the two second actuators 6, in an area 21 of support structure 4 framed by actuators 5, 6, a controllable deformation can be implemented. In this framed area 21, cell culture 12 is placed. Additionally, in framed area 21, sensor device 14 is embedded, by which the force exerted by actuators 5 and 6 on support structure 4 can be detected.

For example, actuators 5 and 6 can be glued with an adhesion-promoting layer 22 onto support structure 4, as is schematically depicted in FIG. 9. It is also possible that with a three-dimensionally structured surface 23 of support structure 4, actuators 5 and 6 can penetrate into an area 24 of support structure 4 close to the surface and thus be secured in form-locked fashion on the surface 23 of support structure 4, as is shown in FIG. 10. Support structure 4 can also have a strip-shaped recess, for example, into which the assigned actuator 5, 6 is partially or completely embedded. It is also conceivable that actuators 5 and 6 consist of a material, or are covered by a material, which has a sufficient force-transmitting adherence to support structure 4, so that actuator 5, 6 does not have to penetrate into support structure 4.

1. A testing arrangement (1, 15, 20) for examination of a cell culture (12) with application of a dynamic force with a support structure (4) for admission of the cell culture (12) and with a force admission device, characterized in that the support structure (4) is configured to be three-dimensional, and is so configured that the cell culture (12) is embedded into the support structure (4), wherein through a force application a deformation of the support structure (4) can be implemented, and that the force admission device has a first actuator (5), that acts on the support structure (4) at a distance from the cell culture (12) embedded in the support structure (4).

2. The testing arrangement (1, 15, 20) of claim 1, characterized in that the force admission device has a second actuator (6), which acts on the support structure (4) at a distance from the cell culture (12) embedded in the support structure (4), and that the first actuator (5) and the second actuator (6) exert a force on the support structure (4) whose directions deviate from each other.

3. The testing arrangement (1) of claim 2, characterized in that the force admission device has a third actuator (7) which acts on the support structure (4) at a distance from the cell culture (12) embedded in the support structure (4), and that the third actuator (7) exerts a force on the support structure (4) whose direction deviates from that of the first actuator (5) and from the second actuator (6).

4. The testing arrangement (1, 15) of one of the foregoing claims, characterized in that the testing arrangement (1) has a holding rack (2) to admit the support structure (4) and that at least one of the actuators (5, 6, 7) is attached to the holding rack (2) in such a way that the actuator (5, 6, 7) can exert a force that deforms the support structure (4) on the support structure (4) admitted in the holding rack (2).

5. The testing arrangement (1) of claim 4, characterized in that the at least one actuator (5, 6, 7) attached on the holding rack (2) is in effective connection with a first side (10) of the support structure (4), and that the support structure (4) is attached on a second side opposite the first side with an attachment device on the holding rack (2), so that through the at least one actuator (5. 6, 7) tensile forces or compression forces can be transferred to the support structure (4).

6. The testing arrangement (1) of claim 5, characterized in that the attachment device, on the second side of the support structure (4) has a further counter-actuator (8), which is attached to the holding rack (2) and can exert a force on the support structure (4).

7. The testing arrangement (1, 15) of one of the foregoing claims 4 to characterized in that the holding rack (2) is configured as a frame rack and at least on two opposite rack sides has transparent openings, through which the support structure (4) can be illuminated and observed.

8. The testing arrangement (1, 15) of one of the foregoing claims, characterized in that the first actuator (5) and if necessary additional actuators (6, 7) and/or counter-actuators (8) are supported so as to be able to shift on the frame rack.

9. The testing arrangement (1, 15, 20) of one of the foregoing claims, characterized in that the support structure (4) has at least one recess (13) into which the first actuator (5) can engage, to effect a deformation of the support structure (4) by application of force.

10. The testing arrangement (1, 15) of claim 9, characterized in that the support structure (4) has at least two recesses (13), which are so arranged at an angle, separated from each other, that a first actuator (5) and a second actuator (6) can each engage into an assigned recess, to engage the support structure (4) and to be able to stand out from a background.

11. The testing arrangement (1, 15) of claim 9 or 10, characterized in that the at least one recess (13) in the support structure (4) is configured to be pocket-like.

12. The testing arrangement (20) of one of the foregoing claims, characterized in that at least one actuator (5, 6) is so arranged and secured on the support structure (4) at a distance from the cell culture (12) that through operation of the actuator (5, 6) the support structure (4) is deformed.

13. The testing arrangement (20) of claim 12, characterized in that multiple actuators (5, 6) surround the cell culture (52) in frame fashion on a surface (23) of the support structure (4).

14. The testing arrangement (1, 15, 20 ) of one of the foregoing claims characterized in that the support structure (4) has fibers.

15. The testing arrangement (1, 15, 20) of one of the forgoing claims, characterized in that in the support structure (4), a biocompatible hydrogel is embedded to admit the cell culture (12) or that the support structure (4) consists of a biocompatible hydrogel.

16. The testing arrangement (1, 15, 20) of one of the foregoing claims, characterized in that at least one of the actuators (5, 6, 7) and/or counter-actuators (8) is a piezoactuator, an electrothermal or electrically active polymer actuator, or an electromechanical, electrochemical, magnetostrictive, hydraulic, pneumatic, bimetallic or electromagnetic actuator.

17. The testing arrangement (1, 15, 20) of one of the foregoing claims, characterized in that at least one of the actuators (5, 6, 7) and/or counter-actuators (8) has a shape-memory material.

18. The testing arrangement (1, 15, 20) of one of the foregoing claims, characterized in that in at least one actuator (5, 6, 7) and/or in at least one counter-actuator (8), and/or in the support structure (4) at least one sensor device (14) is placed for detection of the forces acting on the support structure (4).

19. The testing arrangement (1, 15) according to one of the foregoing claims 4 to 16, characterized in that the holding rack (2) has multiple force admission devices with at least one first actuator (5) and multiple support structures (4), wherein with the multiple first actuators (5) a force is exerted on the support, structure (4) in question that deforms at least one assigned support structure (4).

20. The testing arrangement (1) of claim 19, characterized in that the holding rack (2) has a connection device for connection with a microtiter plate (16), so that the holding rack (2) can be connected, with the microtiter plate (16) in such a way that each support structure (4) is arranged in an assigned cavity (17) of the microtiter plate (16) and can be deformed by the assigned first actuator (5).
